(19) United States
(12) Patent Application Publication
(10) Pub. No.: US 2025/0334566 A1
(43) Pub. Date: Oct. 30, 2025

(54) CALIBRATION OF A NANOPORE ARRAY DEVICE

(71) Applicant: Oxford Nanopore Technologies PLC, Oxford (GB)

(72) Inventors: Daniel Benjamin Trevor Ward, Oxford (GB); Graham James Hall, Oxford (GB)

(73) Assignee: Oxford Nanopore Technologies PLC, Oxford (GB)

(21) Appl. No.: 18/866,109

(22) PCT Filed: May 10, 2023
(86) PCT No.: PCT/GB2023/051219

§ 371 Date: Nov. 15, 2024

(30) Foreign Application Priority Data
May 18, 2022 (GB) 2207267.2

(51) Int. Cl. G01N 33/487; C12Q 1/6869

(52) U.S. Cl. CPC G01N 33/48721; C12Q 1/6869

(57) ABSTRACT

A method of calibrating a nanopore array device is described. The nanopore array device comprises an array of nanopore channels, each nanopore channel formed in a membrane separating two ionic solutions. The nanopore channel connects the ionic solutions and the device further comprises a thermal control component for adjusting the temperature of the array of nanopore channels. The method comprises the steps of measuring signals indicative of ion flow through the nanopore channels; analysing the measurement signals and comparing to a reference value; and adjusting the thermal control component to regulate the temperature of the array of nanopore channels based upon the comparison.

### RELATED APPLICATIONS

This application is a national stage filing under 35 U.S.C. § 371 of international application number PCT/GB2023/051219, filed May 10, 2023, which claims the benefit of United Kingdom application number GB 2207267.2, filed May 18, 2022, each of which is herein incorporated by reference in its entirety.

### BACKGROUND

The present invention relates to a method of calibrating a nanopore device. More particularly the invention relates to a method of calibrating a nanopore array device. Most particularly the invention relates to a method of calibrating a nanopore array device used for sensing molecular entities of an analyte.

The use of nanopores to sense interactions with molecular entities, for example polynucleotides is a powerful technique that has been subject to much recent development. Nanopore devices have been developed that comprise an array of nanopore sensing elements, thereby increasing data collection by allowing plural nanopores to sense interactions in parallel, typically from the same sample.

Nanopore devices may typically employ an electrical signal across a nanopore channel to generate a measurement signal that is interpreted to sense and/or characterise molecular entities as they interact with the nanopore. Typically an electrical signal is applied as a potential difference or current across the array of nanopore channels that will provide a meaningful measurement signal to be interpreted. The measurement can include, for example, one of ionic current flow, electrical resistance, or voltage.

Typically, the electrical signal for the array device is applied to the system at a predetermined value or values. Changes in the measurement signal over time can then be interpreted to determine the molecular entity present in the analyte. However, in an array device, variation across the array in extrinsic factors (i.e. standard conditions) can result in the variation of the measurement signals across the array. In addition, more noticeable variations from standard conditions can occur in devices having multiple flow cells with nanopore channel arrays. In such devices, it can be harder to establish, stabilise and control the standard conditions for each flow cell during an analysis cycle.

In a first embodiment the present invention relates to a method of calibrating a nanopore array device, the nanopore array device comprising an array of nanopore channels, each nanopore channel formed in a membrane separating two ionic solutions, the nanopore channels connecting the ionic solutions, the device further comprising a thermal control component for adjusting the temperature of the array of nanopore channels; the method comprising: measuring signals indicative of ion flow through the nanopore channels; analysing the measurement signals and comparing to a reference value; and adjusting the thermal control component to regulate the temperature of the array of nanopore channels based upon the comparison.

It has been found that variance of controllable conditions such as temperature across the array can result in variation in the measurement signal and this can have a detrimental effect on the measurement confidence and thus overall results generated from the device. Nanopore array devices when used to measure the translocation of a polymer analyte through a nanopore channel typically generate a complex measurement signal dependent upon multiple polymer units, typically having a small range. For example current measurements are typically within the range of 60-120 pA for translocation of a polynucleotide through a protein CsgG nanopore in the presence of 0.5M salt. In addition, alterations in the polymer units may be present that can give rise to even more subtle modulations in the nanopore measurement signal when compared to an unaltered (canonical) polymer unit. An example of such is the methylation of the nucleobase cytosine to form 5-methylcytosine in DNA. In order to analyse the signal and accurately determine the sequence order of the polymer units, various probabilistic and machine learning algorithms have been developed. However, as mentioned above, due to the sensitivity of, and scale of measurements taken by, the device it has been found that even slight variation from standard conditions across the device can have an impact on the measurement signals and therefore the confidence of measurements from the device, for example to accurately predict the sequence order of polymer units.

It has been found that the temperature across the array of nanopore channels in a nanopore array device may vary for a number of reasons. For example, there may be a difference in the ambient temperature of the room or in the device as a whole. More particularly, for a device comprising multiple flow cells with arrays of nanopore channels, there can be a difference in temperature at the individual flow cells. Difference in the temperature in nanopore array devices can also be due to the temperature of the analyte or proximity of the flow cell to componentry within the device that generates heat during operation (for example ASICs and electronic components). Variations in temperature can also exist due to the different intrinsic thermal capacities of the components within the device. Oftentimes, the flow cells are proximate but not connected to each other in the device. This means that there is no direct thermal conduction between the flow cells, which can lead to flow cells experiencing different temperatures.

Calibration of the temperature across the nanopore array device allows for improved normalised signal output and greater confidence in measurements for determining the molecular entities of an analyte. In examples, the reference value may be a value determined by the user or a value held onboard the device and retrieved when required. In this example, the reference value may refer to an idealised value for the analysis of a particular analyte of interest and the nanopore array device can be calibrated accordingly. Alternatively, the reference value may be a value determined from measurements from ion flow through each of the plurality of nanopores (e.g. a mean, modal or median value). In this example, the reference value can be used to calibrate and normalise the rate of ion flow through each of the plurality of nanopores to reduce variations in measurements across the device.

### SUMMARY OF THE INVENTION

Use of an array of nanopores enables the characterisation of an analyte to be carried out with a greater accuracy and speed by combining the measurements signals across the array or multiple arrays. The analyte may for example comprise fragments of a target analyte for example fragments of a target polynucleotide. Alternatively, the analyte being measured may be the target. Data generated from the nanopore array for measurement of the analyte may be combined to increase the accuracy and confidence of the result such as an estimation of the sequence of the analyte or target. In such cases it is advantageous to employ a single algorithm to analyse the measurement signals generated by the multiple nanopores and therefore is it desirable to normalise factors that give rise to variation in the measurement signal in order to optimise the ability of the algorithm to analyse the data.

The thermal control component may comprise an active thermal source such as a heater or a Peltier heater, cooler or heat pump. It may also comprise a passive source such as a heatsink, fan or impeller.

Optionally, the method may contain one or more feedback loops for calibration, in that the method is carried out more than once to adjust the temperature experienced at each flow cell or nanopore channel of the device. The method may also include the optional step of allowing for the thermal control component to set an initial temperature as part of the calibration exercise. In this example the temperature of the device is raised or lowered to an initial state with an expected temperature. The calibration method is then carried out until the device reaches the desired temperature which may or may not include more than one iteration of the method of the present invention.

The signal indicative of ion flow may be measured during translocation of an analyte through the nanopore channels, the measurements signal in this case being indicative of at least the partial translocation of the polymer through a nanopore channel. In addition, each measurement signal may be analysed to determine the speed of translocation of the analyte through the nanopore channels.

The analyte may be a species of interest, or the analyte may be a test analyte of a known composition in order to calibrate the device for subsequent measurement of an analyte of interest. It is understood that reliance on the translocation of an analyte through the nanopore channel for calibration would give more meaning to the measurement signals generated across the array when compared, for example, to an ion flow measurement signal based on open pore current (i.e. flow of electrolyte or ions from the ionic solution through the nanopore).

During translocation of an analyte through a nanopore, the ion current is reduced from an initial value (which may be referred to as the open pore current) which returns to its initial value upon exit of the analyte from the nanopore. The measurement signal may be analysed to determine the time and speed of translocation of the analyte through the nanopore. It has been found that the analysis based on the rate of translocation of an analyte through the nanopore can be correlated roughly to the local temperature of the nanopore system. More particularly, the analyte may be a polymer comprising a sequence of polymer units, and the measurement signal may be analysed to determine the number of polymer units in the sequence and therefore the translocation speed of polymer units/per unit time.

There are various methods for determining the translocation speed (i.e. the rate of translocation of a polymer through a nanopore channel). For example, a polymer having polymer units of known sequence length (namely, the number of polymer units in the polymer is known) may be passed through the nanopore channel to provide a measurement signal in order to determine the time of translocation. The rate of translocation may be compared to a reference rate of translocation and the temperature of the device may be altered to provide either an increase or a decrease in the translocation rate. The user can readily identify from the measurements of the nanopore channel the time between two signals of open-source current, and thus the time taken for the known length of polymer to translocate through the nanopore channel.

In a further example, the polymer having an unknown sequence of polymer units such as an analyte of interest may be used to determine the rate of translocation wherein the measurement signal is analysed to determine the number of bases and therefore the rate of translocation/polymer unit.

In yet a further example, a marker may be provided at a known position to generate a distinct measurement signal from the polymer unit. Suitable examples are the provision of an abasic site in a polynucleotide analyte or one or more non-nucleotides such as a hexacthyleneglycol phosphate spacer. The marker may be advantageously provided in a leader sequence during sample preparation of a polynucleotide analyte of interest. This has the benefit that the rate of translocation may be determined for an analyte of unknown sequence (for example by measuring the time taken to observe the signal from the open pore signal value to the measurement signal due to the marker) without needing to determine the sequence of the polynucleotide analyte. Suitable sample preparation methods to provide a leader sequence for nanopore measurements are disclosed in WO 2015/110813.

The device may be calibrated one or more times during the measurement of the successive translocation of analytes through a nanopore. Preferably the calibration step is carried out at the beginning of the measurement run.

The rate of translocation of the analyte through the nanopore channel may be controlled by an enzyme molecular motor. In examples the polymer may be a polynucleotide, and the enzyme may be a polynucleotide binding protein. Enzyme function is dependent upon temperature and the rate of translocation of the analyte under control of the enzyme will therefore be affected by changes in the local temperature at the nanopore. Typically an increase in temperature results in an increase in translocation speed. Thus the signal may be measured to determine a rate of translocation as an accurate reflection of the temperature experienced by the enzyme molecular motor during translocation of the polymer analyte through the nanopore channel.

As mentioned, there are many factors that can affect the temperature of a nanopore array device and therefore the rate of translocation, such as environmental factors, the temperature of the fluid sample, heat generated by the electronics as well as the different intrinsic thermal capacities of the individual components. Measuring translocation speed and comparing to a reference value advantageously provides a way to calibrate the device wherein the thermal energy provided to the nanopore array may be automatically adjusted without having to directly calculate the local temperature at the nanopore and take into account the variable factors that affect the local temperature

The signal indicative of ion flow may be a current measurement under a potential difference provided across each membrane. The potential difference can be held at a stable value to ensure that any variations in the measurement signals can be more easily related to temperature fluctuations across the device. Alternately, the voltage may be varied during the calibration to ensure that more confidence can be given to any fluctuations in the measurement signals from the device.

The device may comprise a common chamber comprising a common electrode in contact with an ionic solution provided on one side of the membrane, and an array of wells, each well containing an electrode and an ionic solution, each membrane and nanopore channel separating the ionic solution in the common chamber from the ionic solutions contained in each respective well. In this connection, the array of nanopore channels may be provided within a detachable flow cell. The flow cell is typically detachable from the device and the device may have a plurality of flow cells spaced apart from each other in the device, the device may have one or more thermal control components.

The potential difference across the nanopore may be held by reference electrodes provided in respectively the common chamber and in each well. Suitable examples are a soluble redox couple in contact with inert electrode or an Ag/AgCl reference electrode. The potential difference is temperature dependent and a small temperature change, such as 1° C., can have an effect, typically 2%, on the electrode potential. Changes in the potential difference effect the ion flow and therefore the current. Furthermore, for polymers having a charge such as a polynucleotide, a change in potential difference will alter the translocation speed through the nanopore and therefore the measurement signal. Where translocation is controlled for example by an enzyme molecular motor, a change in potential difference may affect the measurement signal for example by changing the force on the bound enzyme-polynucleotide complex.

Each flow cell may have its own dedicated thermal control component. In further examples, the device may comprise a mix of active and passive thermal control component. For example, the device may have its own thermal control component (i.e. a global or macroscale thermal control component such as a fan), as well as a thermal control component for each grouping of flow cells (i.e. a local thermal control component such as a Peltier thermal pump), and also a thermal control component for each flow cell (such as a heat sink). The thermal control component may be placed in close proximity to the flow cell.

A first temperature of the device may be measured and the thermal source is adjusted following the comparison to provide a second device temperature. However knowledge of the temperature is not necessarily required and the thermal source may be adjusted from a first level to a second level. The adjustment is preferably automated and implemented as a result of knowledge of the relative translocation speed. The execution instructions may for example be implemented in software or programmed in hardware such as an FPGA.

The method may be followed by measuring signals indicative of ion flow during translocation of polymer analyte through the array of nanopores and analysing the measurement signals to determine a sequence of polymer units.

In another aspect, the present invention provides a nanopore array device which is configured to perform the method according to the present invention, wherein the steps of the method can be stored in a memory and are implemented in a hardware apparatus or in a computer apparatus. The device may comprise said hardware and/or software apparatus.

The device may comprise of an array of nanopore channels as part of a detachable flow cell. The device may comprise a plurality of detachable flow cells spaced apart from each other. The device may comprise an array of flow cells which are not in direct contact with each other (i.e. spaced apart) which would affect the thermal energy being transferred between flow cells. This presents a challenge when trying to regulate the temperature experienced across the array of flow cells, and the array of nanopore channels in each flow cell. The method allows for a calibration of the temperatures experienced by these flow cells as part of the measurement signal derived from each nanopore channel.

### BRIEF DESCRIPTION OF THE DRAWINGS

To allow better understanding, embodiments of the present invention will now be described by way of non-limitative example with reference to the accompanying drawings, in which:

FIG. 1 is diagram of a nanopore array device;

FIG. 2 is a schematic cross-sectional view of part of a nanopore array device;

FIG. 3 is a schematic of a nanopore array device comprising multiple flow cells;

FIG. 4 is an exploded view of FIG. 3 displaying some of the internal parts if the nanopore array device;

FIG. 5 is perspective view of a nanopore array device;

FIG. 6 is a chart showing the median base-calling rate per flow cell before and after the calibration method of the present invention has been run; and

FIG. 7 is a flow chart of a method performed during the calibration of the nanopore array device of the present invention.

### DETAILED DESCRIPTION

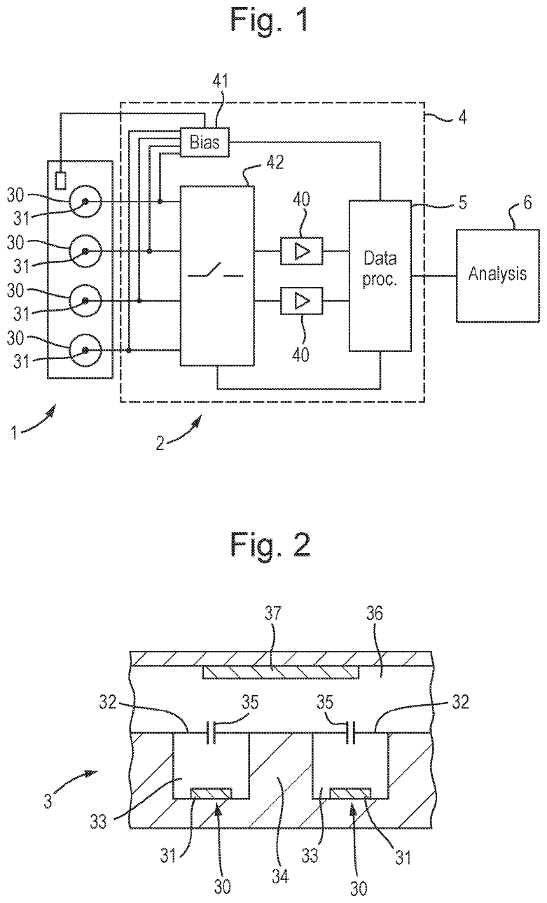

A nanopore array device 1 for sensing interactions of a molecular entities is shown in FIG. 1. The nanopore array device 1 comprises a sensing apparatus 2 comprising a sensor device 3 and a detection circuit 4 that is connected to the sensor device 3.

The sensor device 3 comprises an array of sensing elements 30 that each support respective nanopore channels that are capable of an interaction with a molecular entity. The sensing elements 30 comprise respective electrodes 31. In use, each sensing elements 30 outputs an electrical measurement at its electrode 31 that is dependent on an interaction of a molecular entity with the nanopore. The sensor device 3 is illustrated schematically in FIG. 1 but may have a variety of configurations, some non-limitative examples being as follows.

In one example, the sensor device 3 may have the form shown in FIG. 2. Herein, the sensor device 2 comprises an array of sensing elements 30 which each comprise a membrane 32 supported across a well 33 in a substrate 34 with a nanopore 35 inserted in the membrane 32. The membrane 31 may comprise amphiphilic molecules such as a lipid or a polymer as discussed further below. Each membrane 32 seals the respective well 33 from a sample chamber 36 which extends across the array of sensing elements 30 and is in fluid communication with each nanopore 35. Each well 33 has a sensor electrode 32 arranged therein. A common electrode 37 is provided in the sample chamber 36 for providing a common reference signal (typically a potential or voltage) to each sensor element 30. In use, the sample chamber 36 receives a sample containing molecular entities which interact with the nanopores 35 of the sensing elements 30.

Two sensing elements 30 are shown in FIG. 2 for clarity, but in general any number of sensing elements 30 may be provided. Typically, a large number of sensing elements 30 may be provided to optimise the data collection rate, for example 256, 1024, 4096 or more sensing elements 30.

The sensor device 3 may have a detailed construction as disclosed in WO 2009/077734 or WO 2014/064443 which are herein incorporated by reference in their entireties.

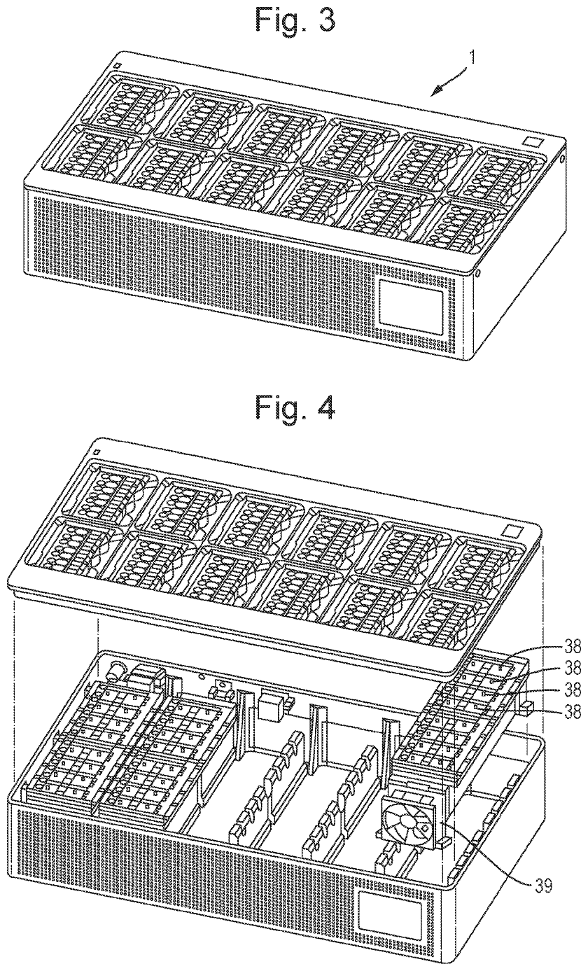

FIGS. 3 to 5 show an example of a nanopore array device 1 comprising a plurality of flow cells 38. Each flow cell 38 comprising a plurality of nanopore channels 25 each supported on a substrate 34 forming at least part of the flow cell 38.

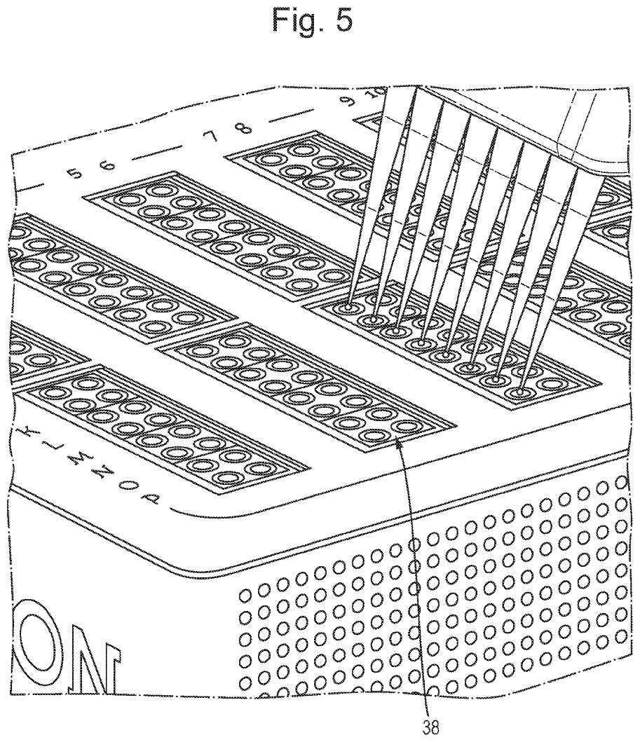

As shown in FIG. 4, the flow cells 38 can be individual components of the nanopore array device 1 and therefore be interchangeable in case of damage or problems with the particular flow cell 38. In the particular device shown in FIGS. 4-5, the flow cells 38 are not formed from on piece of material, and can therefore be said to be not in direct thermal communication with one another. Thus the thermal energy experienced by one flow cell 38 may not relate to the thermal energy of another flow cell 38 in the nanopore array device 1, even if they are neighbouring or in close proximity to one another.

FIG. 4 shows a thermal control component 39 associated with a collection of flow cells 38. The thermal control component 39 in this case is a fan coupled to a collection of flow cells 38 in order to help moderate and regulate the thermal energy they experience in the device. Not shown is a thermal control component that could be coupled to each flow cell 38 in the collection. In addition, there may also be a global thermal control couple for the device as a whole.

The nanopore channels 35 and associated elements of the sensing elements 30 may be as follows, without limitation to the example shown in FIG. 2.

The nanopore channel 35 is a pore, typically having a size of the order of nanometres. In embodiments where the molecular entities are polymers that interact with the nanopore channel 35 while translocating therethrough in which case the nanopore channel 35 is of a suitable size to allow the passage of polymers therethrough.

The nanopore may be a protein pore or a solid-state pore. The dimensions of the pore may be such that only one polymer may translocate the pore at a time.

Where the nanopore is a protein pore, it may have the following properties.

The nanopore may be a transmembrane protein pore. Transmembrane protein pores for use in accordance with the invention include, but are not limited to, β-toxins, such as α-hemolysin, anthrax toxin and leukocidins, and outer membrane proteins/porins of bacteria, such as Mycobacterium smegmatis porin (Msp), for example MspA, lysenin, outer membrane porin F (OmpF), outer membrane porin G (OmpG), outer membrane phospholipase A and Neisseria autotransporter lipoprotein (NalP). α-helix bundle pores comprise a barrel or channel that is formed from α-helices. Suitable α-helix bundle pores include, but are not limited to, inner membrane proteins and a outer membrane proteins, such as WZA and ClyA toxin. The transmembrane pore may be derived from lysenin. The pore may be derived from CsgG, such as disclosed in WO-2016/034591 which is herein incorporated by reference in its entirety. The pore may be a DNA origami pore.

The protein pore may be a naturally occurring pore or may be a mutant pore. The pore may be fully synthetic.

Where the nanopore is a protein pore, it may be inserted into a membrane that is supported in the sensor element 30. Such a membrane may be an amphiphilic layer, for example a lipid bilayer. An amphiphilic layer is a layer formed from amphiphilic molecules, such as phospholipids, which have both hydrophilic and lipophilic properties. The amphiphilic layer may be a monolayer or a bilayer. The amphiphilic layer may be a co-block polymer such as disclosed in WO 2014/064444. Alternatively, a protein pore may be inserted into an aperture provided in a solid-state layer, for example as disclosed in WO 2012/005857.

The nanopore may comprise an aperture formed in a solid-state layer, which may be referred to as a solid-state pore. The aperture may be a well, gap, channel, trench or slit provided in the solid-state layer along or into which analyte may pass. Solid-state layers can be formed from both organic and inorganic materials including, but not limited to, microelectronic materials, insulating materials such as Si₃N₄, Al₂O₃, and SiO, organic and inorganic polymers such as polyamide, plastics such as Teflon® or elastomers such as two-component addition-cure silicone rubber, and glasses. The solid-state layer may be formed from graphene.

Molecular entities interact with the nanopores in the sensing elements 30 causing output an electrical signal at the electrode 31 that is dependent on that interaction.

In one type of sensor device 3, the electrical signal may be the ion current flowing through the nanopore. Similarly, electrical properties other than ion current may be measured. Some examples of alternative types of property include without limitation: ionic current, impedance, a tunnelling property, for example tunnelling current (for example as disclosed in Ivanov AP et al., Nano Lett. 2011 Jan. 12; 11 (1):279-85 which is herein incorporated by reference in its entirety), and a FET (field effect transistor) voltage (for example as disclosed in WO2005/124888 which is herein incorporated by reference in its entirety). One or more optical properties may be used, optionally combined with electrical properties (Soni GV et al., Rev Sci Instrum. 2010 January; 81 (1):014301 which is herein incorporated by reference in its entirety). The property may be a transmembrane current, such as ion current flow through a nanopore. The ion current may typically be the DC ion current, although in principle an alternative is to use the AC current flow (i.e. the magnitude of the AC current flowing under application of an AC voltage).

The interaction may occur during translocation of the molecular entities with respect to the nanopore, for example through the nanopore.

The electrical signal provides as series of measurements of a property that is associated with an interaction between the molecular entity and the nanopore. Such an interaction may occur at a constricted region of the nanopore. For example in the case that the molecular entity is a polymer comprising a series of polymer units which translocate with respect to the nanopore, the measurements may be of a property that depends on the successive polymer units translocating with respect to the pore.

Ionic solutions may be provided on either side of the nanopore. A sample containing the molecular entities of interest that are polymers may be added to one side of the nanopore, for example in the sample chamber 36 in the sensor device of FIG. 2. membrane and allowed to translocate with respect to the nanopore, for example under a potential difference or chemical gradient. The electrical signal may be derived during the translocation of the polymer with respect to the pore, for example taken during translocation of the polymer through the nanopore. The polymer may partially translocate with respect to the nanopore.

In order to allow measurements to be taken as a polymer translocates through a nanopore, the rate of translocation can be controlled by a binding moiety that binds to the polymer. Typically the binding moiety can move a polymer through the nanopore with or against an applied field. The binding moiety can be a molecular motor using for example, in the case where the binding moiety is an enzyme, enzymatic activity, or as a molecular brake. Where the polymer is a polynucleotide there are a number of methods proposed for controlling the rate of translocation including use of polynucleotide binding enzymes. Suitable enzymes for controlling the rate of translocation of polynucleotides include, but are not limited to, polymerases, helicases, exonucleases, single stranded and double stranded binding proteins, and topoisomerases, such as gyrases. For other polymer types, binding moieties that interact with that polymer type can be used. The binding moiety may be any disclosed in WO-2010/086603, WO-2012/107778, and Lieberman KR et al, J Am Chem Soc. 2010; 132 (50) 17961-72), and for voltage gated schemes (Luan B et al., Phys Rev Lett. 2010; 104 (23):238103) which are all herein incorporated by reference in their entireties.

The binding moiety can be used in a number of ways to control the polymer motion. The binding moiety can move the polymer through the nanopore with or against the applied field. The binding moiety can be used as a molecular motor using for example, in the case where the binding moiety is an enzyme, enzymatic activity, or as a molecular brake. The translocation of the polymer may be controlled by a molecular ratchet that controls the movement of the polymer through the pore. The molecular ratchet may be a polymer binding protein.

The polynucleotide handling enzyme may be for example one of the types of polynucleotide handling enzyme described in WO 2015/140535, WO2015/055981 or WO-2010/086603.

Translocation of the polymer through the nanopore may occur, either cis to trans or trans to cis, either with or against an applied potential. The translocation may occur under an applied potential which may control the translocation.

Exonucleases that act progressively or processively on double stranded DNA can be used on the cis side of the pore to feed the remaining single strand through under an applied potential or the trans side under a reverse potential. Likewise, a helicase that unwinds the double stranded DNA can also be used in a similar manner. There are also possibilities for sequencing applications that require strand translocation against an applied potential, but the DNA must be first “caught” by the enzyme under a reverse or no potential. With the potential then switched back following binding the strand will pass cis to trans through the pore and be held in an extended conformation by the current flow. The single strand DNA exonucleases or single strand DNA dependent polymerases can act as molecular motors to pull the recently translocated single strand back through the pore in a controlled stepwise manner, trans to cis, against the applied potential. Alternatively, the single strand DNA dependent polymerases can act as a molecular brake slowing down the movement of a polynucleotide through the pore. Any moieties, techniques or enzymes described in WO-2012/107778 or WO-2012/033524 which are both herein incorporated by reference in their entireties could be used to control polymer motion.

The sensing elements 30 and/or the molecular entities may be adapted to capture molecular entities within a vicinity of the respective nanopores. For example sensing elements 30 may further comprise capture moieties arranged to capture molecular entities within a vicinity of the respective nanopores. The capture moieties may be any of the binding moieties or exonucleases described above with also have the purpose of controlling the translocation or may be separately provided.

The capture moieties may be attached to the nanopores of the sensing elements. At least one capture moiety may be attached to the nanopore of each sensor element.

The capture moiety may be a tag or tether which binds to the molecular entities. In that case the molecular entity may be adapted to achieve that binding.

Such a tag or tether may be attached to the nanopore, for example as disclosed in WO 2018/100370 which is herein incorporated by reference in its entirety, and as further described herein below.

Alternatively in a case the nanopore is inserted in a membrane, such a tag or tether may be attached to the membrane, for example as disclosed in WO 2012/164270 which is herein incorporated by reference in its entirety.

The methods described herein may comprise the use of adapters which adapt the molecular entities for the purpose of capturing them. By way of example, polynucleotide adapters suitable for use in nanopore sequencing of polynucleotides are known in the art. Adapters for use in nanopore sequencing of polynucleotides may comprise at least one single stranded polynucleotide or non-polynucleotide region. For example, Y-adapters for use in nanopore sequencing are known in the art. A Y adapter typically comprises (a) a double stranded region and (b) a single stranded region or a region that is not complementary at the other end. A Y adapter may be described as having an overhang if it comprises a single stranded region. The presence of a non-complementary region in the Y adapter gives the adapter its Y shape since the two strands typically do not hybridise to each other unlike the double stranded portion. The Y adapter may comprise one or more anchors.

The Y adapter preferably comprises a leader sequence which preferentially threads into the pore. The leader sequence typically comprises a polymer. The polymer is preferably negatively charged. The polymer is preferably a polynucleotide, such as DNA or RNA, a modified polynucleotide (such as abasic DNA), PNA, LNA, polyethylene glycol (PEG) or a polypeptide. The leader preferably comprises a polynucleotide and more preferably comprises a single stranded polynucleotide. The adapter may be ligated to a DNA molecule using any method known in the art.

A polynucleotide adapter may comprise a membrane anchor or a transmembrane pore anchor attached to the adapter. For example, a membrane anchor or transmembrane pore anchor may promote localisation of the adapter and coupled polynucleotide within a vicinity of the nanopore. The anchor may be a polypeptide anchor and/or a hydrophobic anchor that can be inserted into the membrane. In one embodiment, the hydrophobic anchor is a lipid, fatty acid, sterol, carbon nanotube, polypeptide, protein or amino acid, for example cholesterol, palmitate or tocopherol.

The anchor may comprise a linker, or 2, 3, 4 or more linkers. Preferred linkers include, but are not limited to, polymers, such as polynucleotides, polyethylene glycols (PEGs), polysaccharides and polypeptides. These linkers may be linear, branched or circular. Suitable linkers are described in WO 2010/086602. Examples of suitable anchors and methods of attaching anchors to adapters are disclosed in WO 2012/164270 and WO 2015/150786 which are both herein incorporated by reference in their entireties.

Examples of tags and tethers which are attached to the nanopore are as follows. Nanopores for use in the methods described herein may be modified to comprise one or more binding sites for binding to one or more analytes (e.g. molecular entities) and thereby acting as a capture moiety. In some embodiments, the nanopores may be modified to comprise one or more binding sites for binding to an adaptor attached to the analytes. For example, in some embodiments, the nanopores may bind to a leader sequence of the adaptor attached to the analytes. In some embodiments, the nanopores may bind to a single stranded sequence in the adaptor attached to the analytes.

In some embodiments, the nanopores are modified to comprise one or more tags or tethers, each tag or tether comprising a binding site for the analyte. In some embodiments, the nanopores are modified to comprise one tag or tether per nanopore, each tag or tether comprising a binding site for the analyte.

In some embodiments, the tag or tether may comprise or be an oligonucleotide.

Other examples of a tag or tether include, but are not limited to His tags, biotin or streptavidin, antibodies that bind to analytes, aptamers that bind to analytes, analyte binding domains such as DNA binding domains (including, e.g., peptide zippers such as leucine zippers, single-stranded DNA binding proteins (SSB)), and any combinations thereof.

The tag or tether may be attached to the external surface of the nanopore, e.g., on the cis side of a membrane, using any methods known in the art. For example, one or more tags or tethers can be attached to the nanopore via one or more cysteines (cysteine linkage), one or more primary amines such as lysines, one or more non-natural amino acids, one or more histidines (His tags), one or more biotin or streptavidin, one or more antibody-based tags, one or more enzyme modification of an epitope (including, e.g., acetyl transferase), and any combinations thereof. Suitable methods for carrying out such modifications are well-known in the art. Suitable non-natural amino acids include, but are not limited to, 4-azido-L-phenylalanine (Faz) and any one of the amino acids numbered 1-71 in FIG. 1 of Liu C. C. and Schultz P. G., Annu. Rev. Biochem., 2010, 79, 413-444 which is herein incorporated by reference in its entirety.

In some embodiments where one or more tags or tethers are attached to the nanopore via cysteine linkage(s), the one or more cysteines can be introduced to one or more monomers that form the nanopore by substitution.

The transmembrane pore may be modified to enhance capture of polynucleotides. For example, the pore may be modified to increase the positive charges within the entrance to the pore and/or within the barrel of the pore. Such modifications are known in the art. For example, WO 2010/055307 discloses mutations in α-hemolysin that increase positive charge within the barrel of the pore.

Modified MspA, lysenin and CsgG pores comprising mutations that enhance polynucleotide capture are disclosed in WO 2012/107778, WO 2013/153359 and WO 2016/034591, respectively which are all herein incorporated by reference in their entireties. Any of the modified pores disclosed in these publications may be used herein.

The arrangement of the detection circuit 4 will now be discussed. The detection circuit 4 is connected to the electrodes 31 of each sensor element 30 and has the primary function of process the electrical signals output therefrom. The detection circuit 4 also has the function of controlling the application of bias signals to each sensor element 30.

The detection circuit 4 includes plural detection channels 40. Each detection channel 40 receives an electrical signal from a single sensor electrode 31 and is arranged to amplify that electrical signal. The detection channel 40 is therefore designed to amplify very small currents with sufficient resolution to detect the characteristic changes caused by the interaction of interest. The detection channel 40 is also designed with a sufficiently high bandwidth to provide the time resolution needed to detect each such interaction. These constraints require sensitive and therefore expensive components. Each detection channel 40 may be similar to standard single channel recording equipment as describe in Stoddart D et al., Proc Natl Acad Sci, 12; 106 (19):7702-7, Lieberman KR et al, J Am Chem Soc. 2010; 132 (50):17961-72, and WO-2000/28312. Alternatively, each detection channel 40 may be arranged as described in detail in WO 2010/122293, WO 2011/067559 or WO 2016/181118.

The analyte of interest to be detected by the nanopore may be a polynucleotide such as DNA or RNA. The analyte may be a polypeptide or a polysaccharide.

The number of sensing elements 30 in the array is greater than the number of detection channels 40 and the nanopore array device is operable to take measurements of a polymer from sensing elements 30 selected in a multiplexed manner, in particular an electrically multiplexed manner. This is achieved by providing a switch arrangement 42 between the sensor electrodes 31 of the sensing elements 30 and the detection channels 40. For clarity, FIG. 1 shows a simplified example with four sensing elements 30 and two detection channels 40, but the number of sensor cells 30 and detection channels 40 is typically much greater. For example, for some applications, the sensor device 2 might comprise a total of 4096 sensing elements 30 and 1024 detection channels 40.

The switch arrangement 42 may be arranged as described in detail in WO 2010/122293. For example, the switch arrangement 42 may comprise plural 1-to-N multiplexers each connected from a detection channel 40 to a group of N sensing elements 30 and may include appropriate hardware such as a latch to select the state of the switching.

By switching of the switch arrangement 42, the nanopore array device 1 may be operated to amplify electrical signals from sensing elements 30 selected in an electrically multiplexed manner. The detection circuit 4 includes a data processor 5 which receives the output signals from the detection channels 40. The data processor 5 acts as a controller that controls the switch arrangement 42 to connect detection channels 40 to respective sensing elements 30 as described further below.

In addition, the detection circuit 4 includes a bias control circuit 41 to perform the function of controlling the application of bias signals to each sensor element 30. The bias control circuit 41 is connected to the common electrode 37 and to the sensor electrodes 31 of each sensor device 30. The bias signals are selected to bias the sensor electrodes 31 with respect to common electrode 37 to control translocation of the molecular entities with respect to the nanopores. In general, it would be possible for a bias signal supplied to a given sensor element 30 to be a drive bias signal that causes translocation to occur at the sensor element 30 or an inhibition bias signal that inhibits translocation to occur at the sensor element 30.

The bias control circuit 41 is controlled by the data processor 5. The data processor has a mode of operation for the bias control circuit 41. Namely, three independent test bias signals are supplied to all the sensing elements 30, thereby causing ionic current flow with respect to the nanopores of each sensing elements 30. The corresponding current flow for each test signal is recorded in the data processor 5 as an amplified electrical signal.

The data processor 5 is arranged as follows. The data processor 5 is connected to the output of the detection channels 40 and is supplied with the amplified electrical signals therefrom. The data processor 5 stores and analyses the amplified electrical signals from the test bias signals to create a calibrated signal. The data processor 5 also controls the other elements of the detection circuit, including control of the bias voltage circuit 41 as described above and control of the switch arrangement 42 as described below. The data processor 5 forms part of the detection circuit 2 and may be provided in a common package therewith, possibly on a common circuit board. The data processor 5 may be implemented in any suitable form, for example as a processor running an appropriate computer program or as an ASIC (application specific integrated circuit).

The data processor 5 of the nanopore array device 1 is connected to an analysis system 6. The data processor 5 also supplies the amplified output signals to the analysis system 6. The analysis system 6 performs further analysis of the amplified electrical signal which is a raw signal representing measurements of the property measured at the nanopore. Such an analysis system 6 may for example estimate the identity of the molecular entity in its entirety or in the case that the molecular entity is a polymer may estimate the identity of the polymer units thereof. Thus, the analysis system may be configured as a computer apparatus running an appropriate program. Such a computer apparatus may be connected to the data processor 5 of the nanopore array device 1 directly or via a network, for example within a cloud-based system.

As can be appreciated, the temperature of the device may not just vary due to variations from standard conditions experience across the nanopore channels 35. The data processor 5 and other electrical components can generate heat which will not be evenly distributed between the nanopore channels 35 on a flow cell 38, or between a plurality of flow cells 38 within said nanopore array device 1.

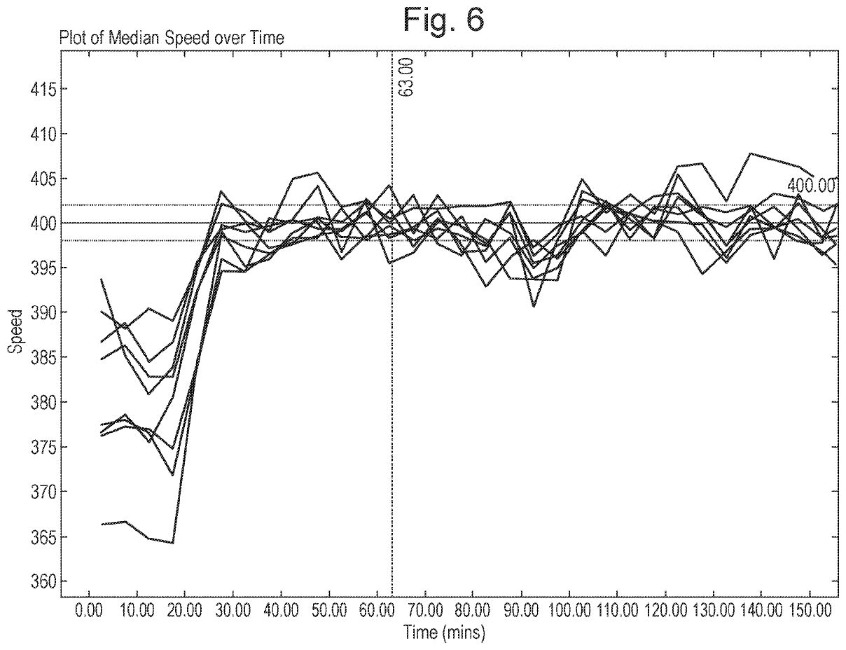

The calibration method of the nanopore array device 1 that is performed by the data processor 5 is shown in FIG. 6 and performed as follows.

The method starts with the performance of a first calibration of the temperature of a flow cell of the nanopore array device 1. For the presently described method, the first calibration is performed by the global thermal control components. However, other thermal control components may also be relied upon such as the fan 39 connected to the collection of flow cells 38 as shown in FIG. 4. Additionally, this initial heating/cooling step may be totally by-passed if the device is already in a ready state.

In the presently described embodiment, a polymer is translocated through the nanopore channel 35 to generate a measurement signal which relates to sequence data. An estimate is provided from a base-caller program being provided on the data processor 5 to translate the measurement signal into an estimate of the rate of translocation of the polymer as it passes through the nanopore channel 35.

The estimated rate of translocated generated by interpreting the measurement signals from the nanopore channels 35 is averaged over the array of nanopore channels 35 per flow cell 38. In a device 1 comprising multiple flow cells 38, the average values of rate of translocation for each flow cell 38 in the device I can be compared and the thermal control component 39 can be adjusted to ensure that the temperature experienced by each flow cell 38 is balanced.

The average rate of translocation is calculated via statistical analysis of the population of rate values for each nanopore channel 35. For example, a mean value could be determined. This mean could be a standard mean, or a weighted mean based on the magnitude of the rate value. Using the standard mean is unfavourable since any skew in the data or outlying rate value(s) will have little influence on the calculated global rate value for a flow cell. In addition it would be complex to attribute correct weighting to rate values to record a true weighted mean for each calibration. Alternatively the mode rate value could be selected as the global offset value. However, this is unfavourable since there may be two or more modes in the rate value data. In another alternative the median rate value could be assigned as the global rate value for the flow cell. This is more most favourable because the median rate value is unaffected by any extreme outliers or non-symmetric distributions of rate values.

The average rates per flow cell are reviewed and compared to a reference value. The reference value can be a number programmed by the user. It may also be an average value of the average rate of translocation provided from each flow cell 38. In this embodiment the reference value is the latter. The average rate from each flow cell 38 is compared to the average rate generated from analysing the average rate from all other flow cells in the device.

If it is found that all flow cells 38 are operating at the required rate (or within a specific deviation from that rate) then the device is considered calibrated and analysis can begin. However, if the rates from one or more of the flow cells 38 is not acceptable then recalibration can occur and the device I can adjust one or more of the temperature control components and the method returns to the first step of calibration, allowing the device to settle in this new state before rates are determined.

The calibration or profiling method of the present invention can be employed prior to the initial use of the nanopore array device 1. Additionally or alternatively, the calibration or method of the present invention can be used once the nanopore array device 1 has been used to sense or detect an analyte of interest.

### EXAMPLE

The following experiment details a method for calibrating a nanopore array device in accordance with the claimed invention.

Eight R9.4.1 PromethION flow cells were provided in a device. Samples comprising a polynucleotide with a leader (poly T) were loaded onto the flow cells. The experiment required live base calling (fast mode) for 24 h to measure the rate of translocation.

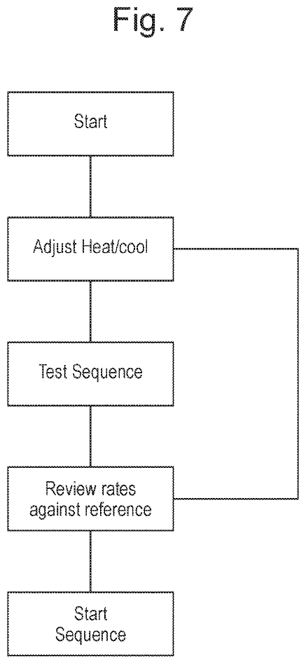

An initial distribution in translocation speeds across the nanopores of the eight flow cells was observed, running analysis through the same nanopore array device, with the same reagents and kit chemistry without calibration. There was a correlation to the flow cell position within the nanopore array device, as well as device-device variations which was considered as being likely due to temperature variations in the fluidic chambers of the flow cell and temperature variations between flow cells (see variance of rate of translocation from the first 20 minutes as shown in FIG. 7).

To provide a normalised rate of translocation at the start of a sequencing run, the measured value of rate of translocation was compared to a reference temperature value and adjusted accordingly (by working out a temperature to speed conversion ratio) to influence speed.

A model was generated using a rate of base translocation to temperature ratio of 30 bases=1° C. The model would measure the temperature and rate of translocation (median) for each position and use that to generate the increase/decrease in temperature required to achieve a target normalised rate of translocation of 400 bps (estimated to be equivalent to 34.5° C.) with a set tolerance level of ±5 bps.

The model was used to adjust and normalise the rate of translocation via increasing or decreasing the temperature via adjustment of thermal control components to obtain the same rate of translocation for each flow cell.

Within the device, the temperature for each flow cell was establish from data recorded from the heat sink and ASIC temp for the duration of the run. A temperature gun using infrared signal to measure the surface temperature of each flow cell was provided to determine the change in temperature of flow cells before and after the calibration had been carried out. The flow cells were calibrated more evenly towards the target temperature of 34.5. The results are shown in the table below.

| Flow | Before speed | After speed |
|---|---|---|
| cell | normalisation (° C.) | normalisation (° C.) |
| 1 | 33.6 ± 0.2 | 34.9 ± 0.2 |
| 2 | 33.8 ± 0.2 | 34.7 ± 0.2 |
| 3 | 32.7 ± 0.2 | 34.2 ± 0.2 |
| 4 | 33.3 ± 0.2 | 34.8 ± 0.2 |
| 5 | 33.2 ± 0.2 | 34.0 ± 0.2 |
| 6 | 33.1 ± 0.2 | 33.9 ± 0.2 |
| 7 | 33.3 ± 0.2 | 34.5 ± 0.2 |
| 8 | 33.0 ± 0.2 | 33.7 ± 0.2 |

The rate of translocation from each flow cell was measured continuously and was seen to converged after the method was performed, with all flow cells reaching a comparable rate of translocation after about 1 hour (63 minutes-see FIG. 7).

The results shown in FIG. 7 suggest a convergence of the median rate of translocation of the flow cells converging once the temperature of the device has been stablised after calibration. The median current was assessed for the eight flow cells to confirm the rate of translocation converge was due to the change in temperatures experienced by the flow cells.

1. A method of calibrating a nanopore array device,
the nanopore array device comprising an array of nanopore channels, each nanopore channel formed in a membrane separating two ionic solutions, the nanopore channel connecting the ionic solutions,
the device further comprising a thermal control component for adjusting the temperature of the array of nanopore channels;
the method comprising:
measuring signals indicative of ion flow through the nanopore channels;
analysing the measurement signals and comparing to a reference value; and
adjusting the thermal control component to regulate the temperature of the array of nanopore channels based upon the comparison.

2. The method according to claim 1, wherein a signal indicative of ion flow is measured during translocation of an analyte through the nanopore channels.

3. The method according to claim 2, wherein each measurement signal is analysed to determine the speed of translocation of the analyte through the nanopore channels.

4. The method according to claim 2 or claim 3, wherein the analyte is a polymer comprising a sequence of polymer units, and wherein the measurements signal are indicative of at least the partial translocation of the polymer through the nanopore channels.

5. The method according to claim 4, wherein each measurement signal is analysed to determine the number of polymer units in the sequence and whereby the average translocation speed of polymer units/per unit time is determined and compared to the reference value.

6. The method according to claim 4 or claim 5, wherein the polymer is a polynucleotide.

7. The method according to any one of claims 4 to 6, wherein the rate of translocation of the polymer through the nanopore channel is controlled by an enzyme molecular motor.

8. The method according to any one of claims 5 to 7 wherein the translocation speed of polymer units/per unit time comprises determining the sequence length of polymer units.

9. The method according to claim 7, wherein the enzyme is a polynucleotide binding protein.

10. The method according to any one of the previous claims, wherein the signal indicative of ion flow through the nanopore channel is a current measurement under a potential difference provided across each membrane.

11. The method according to any one of the previous claims, wherein the device comprises a common chamber comprising a common electrode in contact with an ionic solution provided on one side of the array of nanopore channels and respective membranes, and an array of wells, each well containing an electrode and an ionic solution, each nanopore channel and membrane separating the ionic solution in the common chamber from the ionic solutions contained in each respective well.

12. The method according to any one of claim 11, wherein the array of nanopore channels, the array of wells are provided on a detachable flow cell.

13. The method according to any one of the previous claims, wherein a first temperature of the device is measured and the thermal control component is adjusted from a first level following the comparison to a second level to provide a second device temperature.

14. A method of determining a polymer sequence comprising the step of calibrating the nanopore device according to any one of claims 1 to 13, followed by measuring signals indicative of ion flow during translocation of polymer analyte through the array of nanopore channels and analysing the measurement signals to determine a sequence of polymer units.

15. A nanopore array device configured to perform the method according to any one of the previous claims, wherein the steps of the method are stored in a memory and are implemented in a hardware apparatus or in a computer apparatus.

16. The device according to claim 15 wherein the array of nanopore channels is comprised as part of a detachable flow cell.

17. The device according to claim 16 wherein the detachable flow cell is comprised as part of a plurality of detachable flow cells spaced apart from each other.
